(19) United States
(12) Patent Application Publication
(10) Pub. No.: US 2013/0309284 A1
(43) Pub. Date: Nov. 21, 2013

(54) ORGANIC ULTRAVIOLET ABSORBENT-CONTAINING RESIN PARTICLES, METHOD FOR MANUFACTURING THE SAME, ORGANIC ULTRAVIOLET ABSORBENT-CONTAINING RESIN PARTICLE DISPERSION LIQUID, AQUEOUS DISPERSION ELEMENT, OIL-BASED DISPERSION ELEMENT AND COSMETIC MATERIAL

(71) Applicant: Hirokazu Matsushita, Tokyo (JP)

(72) Inventor: Hirokazu Matsushita, Tokyo (JP)

(73) Assignee: SUMITOMO OSAKA CEMENT CO., LTD., Tokyo (JP)

(21) Appl. No.: 13/981,526

(22) PCT Filed: Jan. 25, 2012
(86) PCT No.: PCT/JP2012/051509

§ 371 Date: Jul. 24, 2013

(30) Foreign Application Priority Data
Jan. 25, 2011 (JP) 2011-012860

(51) Int. Cl. A61K 8/37; A61Q 17/04; A61K 8/02; A61K 8/81; A61K 8/35

(52) U.S. Cl. CPC A61K 8/37; A61K 8/8152; A61K 8/35; A61K 8/0245; A61Q 17/04
USPC 424/401; 424/59

(57) ABSTRACT

The organic ultraviolet absorbent-containing resin particles of the invention contain an organic ultraviolet absorbent and a phosphate surfactant in a resin, in which the average particle diameter of the organic ultraviolet absorbent-containing resin particles is 0.05 μm or more and 1 μm or less, and the content of the organic ultraviolet absorbent is 0.1% by mass or more and 80% by mass or less. According to the organic ultraviolet absorbent-containing resin particles, when the particles are used in a cosmetic material, by the organic ultraviolet absorbent is dispersed in the resin, the ultraviolet-shielding effect is high, the burden for contacted skin can be reduced, and the particles can be safely used. It is not necessary to dissolve the organic ultraviolet absorbent in a specific solvent, the particles can be applied to an aqueous cosmetic material, and the degree of freedom for the formulation as a cosmetic material can be improved.

### TECHNICAL FIELD

The present invention relates to organic ultraviolet absorbent-containing resin particles, a method for manufacturing the same, an organic ultraviolet absorbent-containing resin particle dispersion liquid, an aqueous dispersion element, an oil-based dispersion element and a cosmetic material, and, more specifically, to organic ultraviolet absorbent-containing resin particles preferable for a variety of cosmetic products, such as skin care cosmetic products, makeup cosmetic products and body care cosmetic products, particularly for whitening by skin care cosmetic products, base-making by makeup cosmetic products and sun-screening by body care cosmetic products, for which an ultraviolet-shielding function is required, a method for manufacturing the same, a dispersion liquid, an aqueous dispersion element, an oil-based dispersion element and a cosmetic material including the organic ultraviolet absorbent-containing resin particles.

### BACKGROUND ART

In the past, as an ultraviolet-shielding material used in cosmetic products, there were inorganic ultraviolet-scattering agents and organic ultraviolet absorbents, and a suitable agent has been selectively used depending on use. Examples of the organic ultraviolet absorbent include dibenzoyl methane-based compounds, benzophenone derivatives, para-aminobenzoic acid derivatives, methoxycinnamic acid derivatives, salicylic acid derivatives and the like, and, in particular, as a UVA absorbent that absorbs near-ultraviolet rays, dibenzoyl methane-based compounds, particularly, avobenzone (4-tert-butyl-4′-methoxy dibenzoyl methane) is widely used. Compared with the inorganic ultraviolet-scattering agents, the organic ultraviolet absorbents have advantages that the absorbents do not cause white cast when coated on skin, and exhibits a high transparency.

The organic ultraviolet absorbents chemically absorb the energy of ultraviolet rays to convert into thermal energy, thereby preventing ultraviolet rays from infiltrating into skin cells. As a result, there was a problem in that, when a person with sensitive skin used the organic ultraviolet absorbent, there was a concern of influences on skin, such as the occurrence of allergic symptoms.

In addition, since the organic ultraviolet absorbents exhibit a higher ultraviolet-shielding effect than the inorganic ultraviolet-scattering agents in a small amount, the organic ultraviolet absorbents are used for adults' sunscreens. However, since the organic ultraviolet absorbent is insoluble in water, in order for the absorbent to exhibit the ultraviolet-shielding function, it was necessary to dissolve the agent in a specific solvent, and thus there was a problem in that the degree of freedom for the formulation of a cosmetic product decreased.

In order to solve the above problems, a cosmetic material, in which the organic ultraviolet absorbent is encapsulated in a specific microcapsule so as to prevent the almost absorbent from contacting with skin has been proposed (Patent Citation 1).

### CITATION LIST

### Patent Literature

[Patent Citation 1] Japanese Laid-open Patent Publication No. H07-267841

### SUMMARY OF INVENTION

### Problem to be Solved by the Invention

However, in the above cosmetic material of the related art, since the ultraviolet absorbent was encapsulated in the microcapsule that did not absorb ultraviolet rays, the microcapsule hindered the sufficient absorption of ultraviolet rays, and therefore there was a problem in that the ultraviolet prevention effect is small in consideration of the blending amount of the organic ultraviolet absorbent.

Therefore, when an attempt was made to produce a cosmetic material having a sufficient ultraviolet-shielding effect, there were problems in that it was necessary to relatively increase the blending amount of the organic ultraviolet absorbent and it was difficult to miniaturize the capsule.

The invention has been made in consideration of the above circumstances, and an object of the invention is to provide organic ultraviolet absorbent-containing resin particles which have a high ultraviolet prevention effect, reduces of the organic ultraviolet absorbent to skin contact, and can be blended not only into a water-in-oil (W/O) cosmetic material but also into an oil-in-water (O/W) cosmetic material, a method for manufacturing the same, and a dispersion liquid, an aqueous dispersion element, an oil-based dispersion element and a cosmetic material, in which the organic ultraviolet absorbent-containing resin particles are dispersed.

### Means for Solving the Problem

As a result of repeating thorough studies in order to solve the above problems, the present inventors and the like found that, when an organic ultraviolet absorbent is dissolved in resin monomers including a phosphate surfactant, and the monomers are polymerized to produce fine resin particles, the ultraviolet prevention effect is improved without dissolving the organic ultraviolet absorbent in a specific solvent, and, furthermore, the resin particles can be applied to the formulation of an aqueous cosmetic product, and completed the invention.

That is, organic ultraviolet absorbent-containing resin particles of the invention are organic ultraviolet absorbent-containing resin particles including a resin containing an organic ultraviolet absorbent and a phosphate surfactant, in which an average particle diameter of the organic ultraviolet absorbent-containing resin particles is 0.05 μm or more and 1 μm or less, and a content of the organic ultraviolet absorbent is 0.1% by mass or more and 80% by mass or less.

Organic ultraviolet absorbent-containing resin particles of the invention are organic ultraviolet absorbent-containing resin particles including a resin containing an organic ultraviolet absorbent and a phosphate surfactant, in which an average particle diameter of the organic ultraviolet absorbent-containing resin particles is 0.05 μm or more and 0.5 μm or less, and a content of the organic ultraviolet absorbent is 0.1% by mass or more and 80% by mass or less.

In the organic ultraviolet absorbent-containing resin particles, the organic ultraviolet absorbent is preferably one or two or more selected from the group consisting of dibenzoyl methane-based compounds, benzophenone derivatives, para-aminobenzoic acid derivatives, methoxycinnamic acid derivatives and salicylic acid derivatives.

In a method for manufacturing the organic ultraviolet absorbent-containing resin particles of the invention, an organic ultraviolet absorbent and a phosphate surfactant are dissolved in resin monomers so that a content of the organic ultraviolet absorbent becomes 0.1% by mass or more and 80% by mass or less and a content of the phosphate surfactant becomes 1% by mass or more and 50% by mass or less, thereby producing a resin monomer-dissolved liquid, then, the resin monomer-dissolved liquid is suspended or emulsified in pure water including a suspension protectant, a silicone-based defoamer and a crosslinking agent to produce a suspended liquid or an emulsified liquid, and then a polymerization initiator is added to the suspended liquid or the emulsified liquid to perform suspension polymerization or emulsion polymerization, thereby generating organic ultraviolet absorbent-containing resin particles.

An organic ultraviolet absorbent-containing resin particle dispersion liquid of the invention is an organic ultraviolet absorbent-containing resin particle dispersion liquid with the organic ultraviolet absorbent-containing resin particles of the invention being dispersed in a dispersion medium, in which a content of the organic ultraviolet absorbent-containing resin particles is 1% by mass or more and 80% by mass or less.

An organic ultraviolet absorbent-containing resin particle aqueous dispersion element of the invention is an organic ultraviolet absorbent-containing resin particle aqueous dispersion element with the organic ultraviolet absorbent-containing resin particles of the invention being dispersed in a dispersion medium including alcohols, in which a content of the organic ultraviolet absorbent-containing resin particles is 1% by mass or more and 80% by mass or less, and a content of the alcohols is 5% by mass or more and 20% by mass or less.

An organic ultraviolet absorbent-containing resin particle oil-based dispersion element of the invention is an organic ultraviolet absorbent-containing resin particle oil-based dispersion element with the organic ultraviolet absorbent-containing resin particles of the invention being dispersed in an oily component including a surfactant, in which a content of the organic ultraviolet absorbent-containing resin particles is 1% by mass or more and 80% by mass or less, a content of the oily component is 10% by mass or more and 90% by mass or less, and a content of the surfactant is 1% by mass or more and 40% by mass or less.

A cosmetic material of the invention contains 1% by mass or more and 60% by mass or less of one or two or more selected from the group consisting of the organic ultraviolet absorbent-containing resin particles of the invention, the organic ultraviolet absorbent-containing resin particle dispersion liquid of the invention, the organic ultraviolet absorbent-containing resin particle aqueous dispersion element of the invention and the organic ultraviolet absorbent-containing resin particle oil-based dispersion element of the invention in terms of the organic ultraviolet absorbent-containing resin particles.

### Effects of the Invention

According to the organic ultraviolet absorbent-containing resin particles of the invention, since the average particle diameter of the organic ultraviolet absorbent-containing resin particles is set to 0.05 μm or more and 1 μm or less, and the content of the organic ultraviolet absorbent is set to 0.1% by mass or more and 80% by mass or less by containing the organic ultraviolet absorbent and the phosphate surfactant in the resin, the ultraviolet prevention effect is high, in a case where the organic ultraviolet absorbent-containing resin particles are used for a cosmetic material, the burden for contacted skin can be reduced by dispersing the organic ultraviolet absorbent in the resin, and therefore the particles can be safely used.

In addition, since it is not necessary to dissolve the organic ultraviolet absorbent in a specific solvent, the particles can be applied to an aqueous cosmetic material as well, and it is possible to improve the degree of freedom for the formulation as a cosmetic material.

### BRIEF DESCRIPTION OF DRAWINGS

FIG. 1 is a view illustrating the spectral transmittances of respective resin monomer-dissolved liquids of Examples 1 to 6 of the invention.

FIG. 2 is a view illustrating the volume particle size distribution and cumulative volume particle size distribution of organic ultraviolet absorbent-containing resin particles of Example 2 of the invention.

FIG. 3 is a scanning electron microscopic (SEM) image of the organic ultraviolet absorbent-containing resin particles of Example 2 of the invention.

FIG. 4 is a transmission electron microscopic (TEM) image of the organic ultraviolet absorbent-containing resin particles of Example 2 of the invention.

FIG. 5 is a view illustrating the spectral transmittances of respective moisture gels of Examples 2, 4 to 6 of the invention.

FIG. 6 is a view illustrating the volume particle size distribution and cumulative volume particle size distribution of an organic ultraviolet absorbent-containing resin particle dispersion liquid of Example 4 of the invention.

FIG. 7 is a view illustrating the volume particle size distribution and cumulative volume particle size distribution of an organic ultraviolet absorbent-containing resin particle dispersion liquid of Comparative Example 1.

FIG. 8 is a view illustrating the volume particle size distribution and cumulative volume particle size distribution of an organic ultraviolet absorbent-containing resin particle dispersion liquid of Comparative Example 2.

FIG. 9 is a view illustrating the spectral transmittances of respective simple sunscreens of Examples 7 and 8 and Comparative Examples 3 and 4.

### DESCRIPTION OF EMBODIMENTS

Embodiments of the organic ultraviolet absorbent-containing resin particles, the method for manufacturing the same, the organic ultraviolet absorbent-containing resin particle dispersion liquid, the aqueous dispersion element, the oil-based dispersion element, and the cosmetic material of the invention will be described.

Meanwhile, the following embodiments are to specifically describe the invention in order to make the purpose of the invention well understood, and do not limit the invention unless otherwise designated particularly.

[Organic Ultraviolet Absorbent-Containing Resin Particles]

Organic ultraviolet absorbent-containing resin particles of the present embodiment are organic ultraviolet absorbent-containing resin particles including a resin containing an organic ultraviolet absorbent and a phosphate surfactant (hereinafter sometimes referred to simply as “resin particles”), in which the average particle diameter of the organic ultraviolet absorbent-containing resin particles is 0.05 μm or more and 1 μm or less, and the content of the organic ultraviolet absorbent is 0.1% by mass or more and 80% by mass or less.

Here, the average particle diameter of the organic ultraviolet absorbent-containing resin particles refers to the diameter of particles at the 50 volume % (D50) in a cumulative volume particle size distribution which is obtained by producing a dispersion liquid by dispersing 5% by mass of the organic ultraviolet absorbent-containing resin particles, 10% by mass of polyester-modified silicone and 85% by mass of decamethyl cyclopentasiloxane SH245 (manufactured by Dow Corning Toray Co., Ltd.) using a sand mill through 2500 times of rotation for 3 hours, and measuring the dispersed particle diameters using a dynamic light scattering nanoparticle size analyzer LB-550 (manufactured by Horiba, Ltd.).

Meanwhile, the dispersed particle diameter of D50 obtained using the above measurement method almost coincides with the diameter of the primary particles of the resin particles when observing the organic ultraviolet absorbent-containing resin particles using a scanning electron microscope. Therefore, the average primary particle diameter of the organic ultraviolet absorbent-containing resin particles may be measured as the average particle diameter of the organic ultraviolet absorbent-containing resin particles.

The resin is not particularly limited as long as monomers of the resin can dissolve the organic ultraviolet absorbent, a polymer of the monomers has a high transparency, and can be used as a raw material of the cosmetic material.

Examples of the resin monomers that can be used include (meth)acrylic resins, acrylic acid esters, methacrylic acid esters, acrylic styrene copolymers, acrylic amide copolymers, acrylic epoxy copolymers, acrylic urethane copolymers, acrylic polyester copolymers, silicon acrylic copolymers, vinyl acetate resins, polyamide resins, epoxy resins, urethane resins, polyester resins, silicone resins and the like. Among the above, the monomers of the (meth)acrylic resin are preferable in terms of excellent transparency.

Only one kind of the monomer may be solely polymerized and used, or two or more kinds of the monomers may be combined, polymerized and used.

Examples of the monomers of the (meth)acrylic resin include methyl acrylate, ethyl acrylate, propyl acrylate, n-butyl acrylate, isobutyl acrylate, hexyl acrylate, 2-ethylhexyl acrylate, n-octyl acrylate, dodecyl acrylate, lauryl acrylate, stearyl acrylate, 2-chloroethyl acrylate, phenyl acrylate, methyl α-chloroacrylate, trifluoroethyl acrylate, tetrafluoropropyl acrylate, methyl methacrylate, ethyl methacrylate, propyl methacrylate, n-butyl methacrylate, isobutyl methacrylate, hexyl methacrylate, 2-ethylhexyl methacrylate, n-octyl methacrylate, dodecyl methacrylate, lauryl methacrylate, stearyl methacrylate and the like.

In addition, examples of monomers that can be combined and polymerized with the above acryl include styrene, o-methyl-styrene, m-methyl-styrene, p-methyl-styrene, α-methyl-styrene, o-ethyl-styrene, m-ethyl-styrene, p-ethyl-styrene, 2,4-dimethyl-styrene, p-n-butyl-styrene, p-t-butyl-styrene, p-n-hexyl-styrene, p-n-octyl-styrene, p-n-nonyl-styrene, p-n-decyl-styrene, p-n-dodecyl-styrene, p-methoxy-styrene, p-phenyl-styrene, p-chlorostyrene, 3,4-dichlorostyrene, vinyl acetate, vinyl propionate, vinyl benzoate, vinyl acetate, N-vinylpyrrolidone, vinylidene fluoride, tetrafluoroethylene, hexafluoropropylene, butadiene, isoprene and the like.

Among the above resin monomers, only one kind may be solely polymerized and used, or two or more kinds may be combined, polymerized and used. For example, in a case where the monomer of the (meth)acrylic resin and another monomer are combined, the content of the monomer of the (meth)acrylic resin in the resin monomer is preferably 10% by mass or more and more preferably 30% by mass or more from the viewpoint of transparency.

In addition, an oligomer may be appropriately used.

The organic ultraviolet absorbent is not particularly limited as long as the absorbent can be dissolved in the above resin monomer, and examples thereof include dibenzoyl methane-based compounds, benzophenone derivatives, para-aminobenzoic acid derivatives, methoxycinnamic acid derivatives, salicylic acid derivatives and the like. Among the above organic ultraviolet absorbents, only one kind of absorbent may be solely used, or two or more kinds of absorbents may be combined and used.

The content of the organic ultraviolet absorbent in the resin particles is preferably 0.1% by mass or more and 80% by mass or less, more preferably 0.5% by mass or more and 50% by mass or less, and still more preferably 1% by mass or more and 30% by mass or less.

Here, when the content of the organic ultraviolet absorbent in the resin particles is less than 0.1% by mass, the amount of the organic ultraviolet absorbent is too small to sufficiently develop the ultraviolet-shielding function of the organic ultraviolet absorbent. Consequently, in order to sufficiently develop the ultraviolet-shielding function, a large amount of the resin particles are required, and material design becomes extremely difficult when producing a cosmetic material, which is not preferable. On the other hand, when the content exceeds 80% by mass, the amount of the organic ultraviolet absorbent becomes relatively large with respect to the amount of the resin, consequently, the dispersibility of the organic ultraviolet absorbent in the resin degrades, and the homogeneity of the composition is impaired, which is not preferable.

The average particle diameter of the resin particles is 0.05 μm or more and 1 μm or less, preferably 0.1 μm or more and 0.7 μm or less, more preferably 0.2 μm or more and 0.5 μm or less, and still more preferably 0.2 μm or more and 0.4 μm or less.

Here, when the average particle diameter of the resin particles is less than 0.05 μm, it becomes easy for the resin particles to agglomerate. Therefore, the dispersibility degrades in a case where the resin particles are dispersed in the dispersion medium, and it becomes impossible to sufficiently develop the ultraviolet-shielding function, which is not preferable. Meanwhile, when the average particle diameter exceeds 1 μm, the ultraviolet prevention effect degrades. Furthermore, in a case where the resin particles are used as a cosmetic material, spreading or sliding on skin degrades, and, consequently, a rough feeling or the like is caused to deteriorate a sense of touch or the like, which deteriorates a feeling of using the product, which is not preferable.

The phosphate surfactant is not particularly limited as long as the surfactant is a well-known phosphate or a salt thereof, and polyoxyethylene alkyl phenyl ether phosphate, polyoxyethylene alkyl ether phosphate and salts thereof can be used.

When the phosphate surfactant is included, the emulsion coating strength of the suspended liquid or the emulsified liquid, which will be described below, can increase to accurately control the particle diameter of the resin to be smaller.

[A Method for Manufacturing the Organic Ultraviolet Absorbent-Containing Resin Particles]

The method for manufacturing the organic ultraviolet absorbent-containing resin particles of the embodiment is a method in which the organic ultraviolet absorbent and the phosphate surfactant are dissolved in the resin monomers so that the content of the organic ultraviolet absorbent becomes 0.1% by mass or more and 80% by mass or less and the content of the phosphate surfactant becomes 1% by mass or more and 50% by mass or less, thereby producing a resin monomer-dissolved liquid, then, the resin monomer-dissolved liquid is suspended or emulsified in pure water including a suspension protectant, a silicone-based defoamer and a crosslinking agent to produce a suspended liquid or an emulsified liquid, and then a polymerization initiator is added to the suspended liquid or the emulsified liquid to perform suspension polymerization or emulsion polymerization, thereby generating organic ultraviolet absorbent-containing resin particles.

Here, the method for manufacturing the organic ultraviolet absorbent-containing resin particles will be described in detail.

First, the organic ultraviolet absorbent is dissolved in the resin monomers including the phosphate surfactant to produce a resin monomer-dissolved liquid.

The content of the phosphate surfactant is set to 1% by mass or more and 50% by mass or less with respect to the above resin monomer-dissolved liquid. The reason is that, when the content is less than 1% by mass, the emulsion coating strength of the suspended liquid or the emulsified liquid, which will be described below, does not increase, and, consequently, there are cases where the polymerization efficiency of suspension polymerization or emulsion polymerization decreases. On the other hand, when the content exceeds 50% by mass, the polymerization efficiency cannot be further improved even by increasing any more content, and the phosphate surfactant is wasted.

Meanwhile, an ordinarily-used dispersant may be added as long as the characteristics of the organic ultraviolet absorbent-containing resin particles are not lost.

The dispersant preferably has a high affinity to the resin monomer and a high hydrophobicity.

Examples of the dispersant include carboxylic acids or salts thereof, such as sodium carboxymethyl cellulose, sulfonic acids or salts thereof, such as sodium alkane sulfonic acid, sulfuric acid esters or salts thereof, such as sodium polyoxyethylene nonyl phenyl ether sulfate, and phosphonic acids or salts thereof, such as sodium lauryl phosphonic acid.

Particularly, in a case where the organic ultraviolet absorbent-containing resin particles of the embodiment are used in a cosmetic material, the dispersant should be recognized as a raw material of the cosmetic material at the same time.

The method for dissolving the organic ultraviolet absorbent in the resin monomers is not particularly limited as long as the organic ultraviolet absorbent is dissolved in the resin monomer, and an appropriate mixing method may be selected in consideration of the compatibility of the resin monomers and the organic ultraviolet absorbent.

Regarding the degree of mixing, the organic ultraviolet absorbent needs to be mixed until the sedimentation of a solid content cannot be visually confirmed. Examples of a mixing apparatus used in the mixing process include a magnetic stirrer, a stirrer or the like.

Thereby, the resin monomer-dissolved liquid containing the organic ultraviolet absorbent can be obtained.

Next, the resin monomer-dissolved liquid is suspended or emulsified in pure water including a suspension protectant, a silicone-based defoamer and a crosslinking agent to produce a suspended liquid or an emulsified liquid having a dispersed particle diameter of 0.05 μm or more and 1 μm or less.

Examples of the suspension protectant include non-ionic surfactants, such as polyoxyethylene alkyl ethers and polyoxy ethylene alkyl phenyl ethers, anionic surfactants, such as alkyl benzene sulfonic acid salts, alkyl sulfuric acid ester salts and alkyl phenyl sulfuric acid esters, and the like, and, among the above, the anionic surfactants are preferable, and the anionic surfactant is preferably an alkyl benzene sulfonic acid salt.

The addition amount of the suspension protectant is preferably 0.1% by mass or more and 10% by mass or less, and more preferably 0.1% by mass or more and 2% by mass or less with respect to the above resin monomer-dissolved liquid.

Examples of the silicone-based defoamer include oil-type defoamers, oil compound-type defoamers, solution-type defoamers, powder-type defoamers, solid solution-type defoamers, emulsion-type defoamers, self-emulsification-type defoamers and the like, and, among the above, the oil compound-type defoamers are preferable.

The addition amount of the silicone-based defoamer is preferably 0.01% by mass or more and 5% by mass or less and more preferably 0.1% by mass or more and 1% by mass or less with respect to the above resin monomer-dissolved liquid.

When 0.01% by mass or more and 5% by mass or less of the silicone-based defoamer is added with respect to the above resin monomer-dissolved liquid, the stirring rate of a mixer, a stirrer, a homomixer, a homogenizer or the like can be significantly increased, and, consequently, the resin particles can be made to be as small as approximately 50 nm.

Therefore, in a case where the resin particles are applied to a cosmetic material or the like, it is possible to provide a cosmetic material having superior transparency and an excellent feeling of using the product with no rough feeling.

In addition, the stirring rate of a mixer, a stirrer, a homomixer, a homogenizer or the like can be significantly increased, and, consequently, it is possible to improve the manufacturing efficiency and yield of the organic ultraviolet absorbent-containing resin particles, and therefore the manufacturing cost can be significantly reduced.

The crosslinking agent is not particularly limited as long as the crosslinking agent is a monomer having two or more unsaturated double bonds, and can be appropriately selected from polyfunctional vinyl monomers, polyfunctional (meth)acrylic acid ester derivatives and the like.

More specific examples thereof include (poly)alkylene glycol-based di(meth)acrylates, such as divinyl benzene, divinyl biphenyl, divinyl naphthalene, (poly)ethylene glycol di(meth)acrylate, (poly)propylene glycol di(meth)acrylate and (poly)tetramethylene glycol di(meth)acrylate.

In addition, examples thereof include alkanediol-based di(meth)acrylates, such as 1,6-hexanediol di(meth)acrylate, 1,8-octanediol di(meth)acrylate, 1,9-nonanediol di(meth)acrylate, 1,10-decanediol di(meth)acrylate, 1,12-dodecandiol di(meth)acrylate, 3-methyl-1,5-pentanediol di(meth)acrylate, 2,4-diethyl-1,5-pentanediol di(meth)acrylate, butyl ethyl propanediol di(meth)acrylate, 3-methyl-1,7-octanediol di(meth)acrylate and 2-methyl-1,8-octanediol di(meth)acrylate.

In addition, examples thereof include neopentyl glycol di(meth)acrylates, trimethylolpropane tri(meth)acrylates, tetramethylolmethane tri(meth)acrylates, tetramethylol propane tetra(meth)acrylates, pentaerythritol tri(meth)acrylates, ethoxified cyclohexane dimethanol di(meth)acrylates, ethoxified bisphenol A di(meth)acrylates, tricyclodecane dimethanol di(meth)acrylates, propoxified ethoxified bisphenol A di(meth)acrylates, 1,1,1-tris hydroxylmethyl ethane di(meth)acrylates, 1,1,1-tris hydroxylmethyl ethane tri(meth)acrylates, 1,1,1-tris hydroxylmethyl propane triacrylates, diaryl phthalates and isomers thereof, triaryl isocyanuate and derivatives thereof, and the like.

Among the above, (poly)ethylene glycol di(meth)acrylates are particularly preferable.

The addition amount of the crosslinking agent is preferably 0.1% by mass or more and 10% by mass or less, and more preferably 1% by mass or more and 10% by mass or less with respect to the above resin monomer-dissolved liquid.

Next, a polymerization initiator is added to the above suspended liquid or the emulsified liquid, and suspension polymerization or emulsion polymerization is performed.

Examples of the polymerization initiator include salts of persulfuric acid, such as potassium persulfate and ammonium persulfate; organic peroxides, such as hydrogen peroxide, benzoyl peroxide, lauroyl peroxide, t-butyl hydroperoxide, benzoyl peroxide and cumene hydroperoxide; azo-based polymerization initiators, such as azobisdiisobutyronitrile and 2,2-azobis(2-amidinopropane)dihydrochloride; and the like, and, among the above, the salts of persulfuric acid are preferable.

The addition amount of the polymerization initiator is preferably 0.01% by mass or more and 1% by mass or less and more preferably 0.05% by mass or more and 0.5% by mass or less with respect to the above suspended liquid or the emulsified liquid.

In the polymerization method, polymerization is preferably carried out by stirring and heating the above suspended liquid or the emulsified liquid in a nitrogen atmosphere in the presence of the polymerization initiator.

The polymerization initiating temperature is preferably set to 50° C. or more and 80° C. or less. In addition, the holding time at the above temperature is preferably approximately 1 hour to 5 hours, and an appropriate time may be selected in consideration of a period of time, during which unreacted residual monomers become the minimum, the polymerization state and the manufacturing cost.

After that, the liquid is cooled using ice or naturally cooled, and the polymerization reaction is stopped.

When the content of the suspension protectant, the silicone-based defoamer and the polymerization initiator is limited to the above range in the suspension polymerization and the emulsion polymerization, it is possible to control the average particle diameter of the obtained organic ultraviolet absorbent-containing particles to 0.05 μm or more and 1 μm or less.

Next, in order to remove the residual monomer, polymerization initiator and surfactant from the obtained polymer, the polymer is sufficiently washed using an alcohol, and then washed using pure water.

The alcohol is preferably an alcohol that is soluble in pure water and is easily washed away, examples thereof include ethanol, 2-propanol and the like, and 2-propanol is particularly preferable.

The washing method is not particularly limited as long as the residual monomers and the like can be removed, and the polymer is washed using pressure filtration, suction filtration, filter press, centrifugal separation, ultrafiltration, decantation or the like. For example, in a case where the polymer is washed using 2-propanol, washing is carried out until the concentration of 2-propanol becomes 1% or less, and the conductivity becomes 20 μS/cm or less.

After the completion of the washing, the obtained polymer is dried at 80° C. to 100° C. to remove the alcohol or the pure water, and then the obtained polymer is ground. The drying method is not particularly limited as long as the alcohol or the pure water can be removed, and examples thereof include drying at atmospheric pressure, drying in a vacuum and the like.

The grinding method is not particularly limited as long as the obtained polymer can be ground into particles of 0.05 μm or more and 1 μm or less, and examples thereof include with a pin mill, a hammer mill, a jet mill, an impeller mill and the like.

Thereby, the organic ultraviolet absorbent-containing resin particles can be generated.

When the respective particles agglomerated by the drying are ground by the ultraviolet-shielding composite particles going through a grinding process, it is possible to improve a feeling of using the product in a case where the particles are used in a cosmetic material.

[Organic Ultraviolet Absorbent-Containing Resin Particle Dispersion Liquid]

An organic ultraviolet absorbent-containing resin particle dispersion liquid of the embodiment is a dispersion liquid with the above organic ultraviolet absorbent-containing resin particles being dispersed in a dispersion medium, in which the content of the organic ultraviolet absorbent-containing resin particles is 1% by mass or more and 80% by mass or less, more preferably 20% by mass or more and 70% by mass or less, and still more preferably 30% by mass or more and 60% by mass or less.

The dispersion medium may be a solvent that can disperse the above organic ultraviolet absorbent-containing resin particles, and examples thereof that can be preferably used include, in addition to water, alcohols, such as methanol, ethanol, 2-propanol, butanol and octanol; esters, such as ethyl acetate, butyl acetate, ethyl lactate, propylene glycol monomethyl ether acetate, propylene glycol monoethyl ether acetate and γ-butyrolactone; ethers, such as diethyl ether, ethylene glycol monomethyl ether (methylcellosolve), ethylene glycol monoethyl ether (ethyl cellosolve), ethylene glycol monobutyl ether (butyl cellosolve), diethylene glycol monomethyl ether and diethylene glycol monoethyl ether; ketones, such as acetone, methyl ethyl ketone, methyl isobutyl ketone, acetylacetone and cyclohexanone; aromatic hydrocarbons, such as benzene, toluene, xylene and ethylbenzene; amides, such as dimethyl formamide, N,N-dimethyl aceteamide and N-methylpyrrolidone; chain-like polysiloxanes, such as dimethylpolysiloxane, methyl phenyl polysiloxane and diphenyl polysiloxane; cyclic polysiloxanes, such as octamethyl cyclotetrasiloxane, decamethylcyclopentasiloxane and dodecamethylcyclohexanesiloxane; modified polysiloxanes, such as amino-modified polysiloxane, polyether-modified polysiloxane, alkyl-modified polysiloxane and fluorine-modified polysiloxane. Only one of the above solvents or a mixture of two or more can be used.

Here, when the content of the organic ultraviolet absorbent-containing resin particles is less than 1% by mass, the amount of the organic ultraviolet absorbent becomes too small, for the dispersion liquid to sufficiently exhibit the ultraviolet-shielding function, and, consequently, material design becomes extremely difficult when producing a cosmetic material, which is not preferable. On the other hand, when the content exceeds 80% by mass, the amount of the organic ultraviolet absorbent in the dispersion liquid becomes relatively large, consequently, the dispersibility of the organic ultraviolet absorbent in the dispersion liquid decreases, and the homogeneity of the composition is impaired, which is not preferable.

The dispersion liquid can be obtained by mixing the above organic ultraviolet absorbent-containing resin particles with the dispersion medium, mixing the dispersant or an aqueous binder as necessary, then, performing a dispersion treatment on the mixture using a disperser or a mixer, such as a beads mill, a ball mill or a homogenizer in which a sand mill and zirconia beads are used, and dispersing the organic ultraviolet absorbent-containing resin particles in the dispersion medium.

In addition, the necessary time for the dispersion treatment is not particularly limited as long as the time is long enough for the organic ultraviolet absorbent-containing resin particles to be dispersed in the dispersion medium.

[Organic Ultraviolet Absorbent-Containing Resin Particle Aqueous Dispersion Element]

An organic ultraviolet absorbent-containing resin particle aqueous dispersion element of the embodiment is an aqueous dispersion element including 1% by mass or more and 80% by mass or less, more preferably 20% by mass or more and 70% by mass or less, and still more preferably 30% by mass or more and 60% by mass or less of the above organic ultraviolet absorbent-containing resin particles and 5% by mass or more and 20% by mass or less of the alcohol.

The aqueous dispersion element may further contain 0.001% by mass or more and 10% by mass or less, more preferably 0.005% by mass or more and 5% by mass or less and still more preferably 0.01% by mass or more to 3% by mass or less of a water-soluble macromolecule. In this case, it is necessary to adjust the contents of the respective components so that the total of the contents of the respective components of the organic ultraviolet absorbent-containing resin particles, the alcohol and the water-soluble macromolecule does not exceed 100% by mass.

Examples of the alcohol include monovalent alcohols or polyvalent alcohols having 1 to 6 carbon atoms, such as ethanol, 1-propanol, 2-propanol, 1-butanol, 2-butanol, glycerin, 1,3-butylene glycol, propylene glycol and sorbitol, and, among the above, a monovalent alcohol, particularly, ethanol is preferable.

In a case where the aqueous dispersion element does not include the water-soluble macromolecule, the content of the alcohol is preferably 5% by mass or more and 20% by mass or less, and more preferably 10% by mass or more and 20% by mass or less.

Particularly, in a case where the content of the alcohol is set to 10% by mass or more and 20% by mass or less, it is possible to improve the dispersibility of the organic ultraviolet absorbent-containing resin particles and the stability of the aqueous dispersion element over time, which is preferable.

In addition, in a case where the aqueous dispersion element includes the water-soluble macromolecule, the water-soluble macromolecule is not particularly limited as long as the water-soluble macromolecule can be used for a cosmetic product, and examples thereof include gum arabic, sodium alginate, casein, carrageenan, galactan, carboxyvinyl polymers, carboxymethyl cellulose, sodium carboxymethyl cellulose, carboxymethyl starch, agar, xanthan gum, quince seed, guar gum, collagen, gelatin, cellulose, dextran, dextrin, tragacanth gum, hydroxyl ethyl cellulose, hydroxyl propyl cellulose, sodium hyaluronate pectin, pullulan, methyl cellulose, methyl hydroxypropyl cellulose and the like. Only one of the aqueous macromolecules may be solely used, or a mixture of two or more may be used.

The water-soluble macromolecule plays a role of a dispersant and a viscosity adjuster, and the addition of the water-soluble macromolecule improves the dispersibility of the organic ultraviolet absorbent-containing resin particles and the stability of the aqueous dispersion element over time.

In a case where the aqueous dispersion element includes the water-soluble macromolecule, the content of the alcohol is preferably 5% by mass or more and 20% by mass or less, and more preferably 15% by mass or more and 20% by mass or less.

Here, the reason for setting the content of the alcohol to 5% by mass or more and 20% by mass or less, in a case where the aqueous dispersion element includes the aqueous macromolecule, is that, when the content is less than 5% by mass, the content of the alcohol is too small, therefore, the aqueous macromolecule cannot evenly infiltrate into the alcohol and unevenly swells due to moisture, consequently, the dispersibility of the organic ultraviolet absorbent-containing resin particles decreases to be difficult for handling, and, furthermore, the stability of the aqueous dispersion element over time degrades, which is not preferable. In addition, when the content exceeds 20% by mass, the viscosity of the entire aqueous dispersion element becomes high, the dispersion stability of the organic ultraviolet absorbent-containing resin particles degrades, and the stability of the aqueous dispersion element over time also degrades, which is not preferable.

That is, when the content of the alcohol is adjusted within the above range, it is possible to obtain an organic ultraviolet absorbent-containing resin particle aqueous dispersion element that can ensure sufficient transparency in a case where the dispersion element is spread and coated on skin.

The organic ultraviolet absorbent-containing resin particle aqueous dispersion element can be obtained by, for example, mixing the above organic ultraviolet absorbent-containing resin particles with a solvent including the alcohol or a mixture including the alcohol and the water-soluble macromolecule, and then mixing water with the mixture to disperse the particles. The amount of the water may be appropriately adjusted, and is preferably within a range of 15% by mass or more and 94% by mass or less.

The mixing method is not particularly limited, and the organic ultraviolet absorbent-containing resin particle aqueous dispersion element can be obtained by appropriately dispersing the organic ultraviolet absorbent-containing resin particles using a disperser or a mixer, such as a beads mill, a ball mill or a homogenizer in which a sand mill and zirconia beads are used.

[Organic Ultraviolet Absorbent-Containing Resin Particle Oil-Based Dispersion Element]

An organic ultraviolet absorbent-containing resin particle oil-based dispersion element of the embodiment is an organic ultraviolet absorbent-containing resin particle oil-based dispersion element with the above organic ultraviolet absorbent-containing resin particles being dispersed in an oily component including a surfactant, in which the content of the organic ultraviolet absorbent-containing resin particles is 1% by mass or more and 80% by mass or less, the content of the oily component is 10% by mass or more and 90% by mass or less, and the content of the surfactant is 1% by mass or more and 40% by mass or less.

The content of the organic ultraviolet absorbent-containing resin particles is preferably 20% by mass or more and 70% by mass or less, and more preferably 30% by mass or more and 60% by mass or less.

When the content of the organic ultraviolet absorbent-containing resin particles is adjusted within the above range, it is possible to obtain an organic ultraviolet absorbent-containing resin particle aqueous dispersion element that can ensure sufficient transparency in a case where the dispersion element is spread and coated on skin.

The oily component is not particularly limited as long as the component can be used for a cosmetic product, and examples thereof include hydrocarbons, fatty oils, waxes, hardened oils, ester oils, aliphatic acids, higher alcohols, silicone oils, fluorine-based oils, lanoline derivatives, oily gelators and the like. The oily component may be derived from any of animal oils, plant oils, synthetic oils and the like, and may have any properties of solid oils, semisolid oils, liquid oils, volatile oils and the like.

Examples of the hydrocarbons include liquid paraffins, squalene, Vaseline, polyethylene waxes, ethylene.propylene copolymers, paraffin waxes, montan waxes, Fischer-Tropsch waxes, polyisobutylene, polybutene, ceresin waxes, ozokerite waxes and the like.

Examples of the fatty oils include Japan waxes, olive oil, castor oil, mink oil, macadamia oil and the like.

Examples of the waxes include beeswax, spermaceti, Carnauba wax, candelilia wax and the like.

Examples of the ester oils include jojoba oil, glyceryl trioctanoin acid, polyglyceryl diisostearic acid, diclyceryl triisostearic acid, glyceryl tribehenate, cetyl 2-ethylhexanoic acid, isopropyl myristate, isopropyl palmitate, octyldodecyl myritate, pentaerythrityl rosinate, neopentyl glycol dioctanoate, cholesterol fatty acid ester, phytosterol fatty acid esters, triglycerides, diisostearyl malate and the like.

Examples of the aliphatic acids include stearic acid, lauric acid, myristic acid, behenic acid, isostearic acid, oleic acid and the like.

Examples of the higher alcohols include stearyl alcohol, cetyl alcohol, lauryl alcohol, oleyl alcohol, isostearyl alcohol, behenyl alcohol and the like.

Examples of the silicone oils include chain-like silicones, such as dimethyl polysiloxane and methyl phenyl polysiloxane; cyclic silicones, such as octamethylcyclotetrasiloxane and decamethylcyclopentasiloxane; modified silicones, such as crosslinking polyether-modified methyl polysiloxane, methacryl-modified organopolysiloxane, stearyl-modified organopolysiloxane, oleyl-modified organopolysiloxane, behenyl-modified organopolysiloxane, alkoxy-modified organopolysiloxane, fluorine-modified organopolysiloxane, fluorine-modified polysiloxane, amino-modified polysiloxane, fatty acid-modified polysiloxane and higher alcohol-modified polysiloxane; methyl phenyl polysiloxane having a high degree of polymerization, dimethyl polysiloxane having a high degree of polymerization, crosslinking organopolysiloxane and the like.

Only one of the silicone oils may be solely used, or a mixture of two or more may be used.

Among the above silicone oils, a chain-like silicone having a low molecular weight of 20000 or less or a volatile silicone is preferable since the silicone oils have a low surface tension and is thus smoothly spread when coated on skin. In addition, since the silicone oils have a smooth feeling, a rough feeling or oiliness is reduced, and a fresh feeling of using a cosmetic product can be obtained, which is preferable.

Examples of the fluorine-based oils include perfluorodecane, perfluorooctane, perfluoropolyether and the like.

Examples of the lanoline derivatives include lanoline, lanoline acetate, lanoline fatty acid isopropyl ester, lanoline alcohols and the like.

Examples of the oily gelators include sucrose fatty acid ester, starch fatty acid ester, aluminum isostearate, 1,2-hydroxy stearic acid and the like.

Only one of the above oily components may be solely used or a mixture of two or more may be used.

Among the above oily components, the silicone oil is preferable since the silicone oil reduces a sticky feeling or oiliness, and a fresh feeling of using the product can be obtained.

The surfactant is not particularly limited as long as the surfactant can be used for cosmetic products, and examples thereof include non-ionic surfactants, anionic surfactants, cationic surfactants, ampholytic surfactants and the like.

Examples of the non-ionic surfactants include glycerin fatty acid esters and alkylene glycol adducts thereof, polyglycerin fatty acid esters and alkylene glycol adducts thereof, propylene glycol fatty acid esters and alkylene glycol adducts thereof, sorbitan fatty acid esters and alkylene glycol adducts thereof, fatty acid esters of sorbitol and alkylene glycol adducts thereof, polyalkylene glycol fatty acid esters, sucrose fatty acid esters, polyoxyl alkylene alkyl esters, glycerine alkyl ethers, polyoxyethylene alkyl phenyl ethers, polyoxyethylene cured castor oil, alkylene glycol adducts of lanoline, poloxyalkylene alkyl-comodified silicones, polyether-modified silicones and the like.

Examples of the anionic surfactants include inorganic and organic salts of fatty acids, such as stearic acid and lauric acid, alkyl benzene sulfate, alkyl sulfonate, α-olefin sulfonate, dialkyl sulfosuccinate, α-sulfonated fatty acid salts, acyl methyl taurate, N-methyl-N-alkyl taurate, polyoxyethylene alkyl ether sulfate, polyoxyethylene alkyl phenyl ether sulfate, alkyl phosphate, polyoxyethylene alkyl ether phosphate, polyoxyethylene alkyl phenyl ether phosphate, N-acyl aminoate, N-acyl-N-alkyl aminoate, o-alkyl-substituted malate, alkyl sulfosuccinate and the like.

Examples of the cationic surfactants include alkyl amine salts, polyamine and alkanolamine fatty acid derivatives, alkyl quaternary ammonium salts, cyclic quaternary ammonium salts, and the like.

Examples of the ampholytic surfactants include carbonic acid-type surfactants, sulfate surfactants, sulfonate surfactants, phosphate surfactants and the like.

Only one of the surfactants maybe solely used, or a mixture of two or more may be used. Among the surfactants, a non-ionic surfactant is preferable, and, in particular, polyether-modified silicone is preferable since the dispersibility is favorable, and it is possible to obtain a cosmetic material having excellent adhesion to skin.

The content of the surfactant in the oil-based dispersion element is preferably 1% by mass or more and 40% by mass or less, and more preferably 10% by mass or more and 30% by mass or less with respect to the organic ultraviolet absorbent-containing resin particles.

Here, the reason for setting the content of the surfactant in the oil-based dispersion element to 1% by mass or more and 40% by mass or less is that, when the content is less than 1% by mass, since the content of the surfactant is too small, the surfactant cannot evenly infiltrate onto the surfaces of the organic ultraviolet absorbent-containing resin particles, consequently, the dispersibility of the organic ultraviolet absorbent-containing resin particles degrades to be difficult for handling, and, furthermore, the stability of the oil-based dispersion element over time degrades, which is not preferable. In addition, when the content exceeds 40% by mass, the viscosity of the entire oil-based dispersion element becomes high, the dispersion stability of the organic ultraviolet absorbent-containing resin particles degrades, the stability of the oil-based dispersion element over time degrades, and a sticky feeling or oiliness increases, which is not preferable.

The organic ultraviolet absorbent-containing resin particle oil-based dispersion element can be obtained by mixing the above organic ultraviolet absorbent-containing resin particles with a mixture (dispersion medium) including a solvent including the oily component and the surfactant.

The mixing method is not particularly limited, and the organic ultraviolet absorbent-containing resin particle oil-based dispersion element can be obtained by performing a dispersion treatment using a disperser or a mixer, such as a beads mill, a ball mill or a homogenizer in which a sand mill and zirconia beads are used, and dispersing the organic ultraviolet absorbent-containing resin particles in the dispersion medium.

In addition, the necessary time for the dispersion treatment is not particularly limited as long as the time is long enough for the organic ultraviolet absorbent-containing resin particles to be dispersed in the dispersion medium.

[Cosmetic Material]

A cosmetic material of the embodiment is a cosmetic material including 1% by mass or more and 60% by mass or less of one or two or more selected from the group consisting of the organic ultraviolet absorbent-containing resin particles, the organic ultraviolet absorbent-containing resin particle dispersion liquid, the organic ultraviolet absorbent-containing resin particle aqueous dispersion element and the organic ultraviolet absorbent-containing resin particle oil-based dispersion element in terms of the organic ultraviolet absorbent-containing resin particles, and, when containing the organic ultraviolet absorbent-containing resin particles within the above range, the cosmetic material becomes transparent and has an excellent feeling of using the product with no rough feeling.

The cosmetic material can be obtained by blending one or two or more selected from the group consisting of the organic ultraviolet absorbent-containing resin particles, the organic ultraviolet absorbent-containing resin particle dispersion liquid, the organic ultraviolet absorbent-containing resin particle aqueous dispersion element and the organic ultraviolet absorbent-containing resin particle oil-based dispersion element into a milky liquid, a cream, a foundation, a lip stick, a blush, an eye shadow or the like as blended in the related art.

Furthermore, when the cosmetic material of the embodiment is used as a component of a cosmetic product, it is possible to provide a variety of cosmetic products, such as skin care cosmetic products, makeup cosmetic products and body care cosmetic products, which are excellent in terms of transparency and a feeling of using the product. Particularly, in a case where the cosmetic material is used for whitening by skin care cosmetic products, base-making by makeup cosmetic products and sun-screening by body care cosmetic products, for which an ultraviolet-shielding function is required, it is possible to provide a cosmetic product which is excellent in terms of the ultraviolet-shielding function, transparency and a feeling of using the product.

As described above, according to the organic ultraviolet absorbent-containing resin particles of the embodiment, since the organic ultraviolet absorbent-containing resin particles are evenly dispersed in the resin by dissolving the organic ultraviolet absorbent in the resin monomer, it is possible to reduce the burden caused by the ultraviolet absorbent to skin.

In addition, since it is not necessary to dissolve the ultraviolet absorbent in a specific solvent, the ultraviolet absorbent-containing resin particles of the embodiment can be blended not only into a water-in-oil (W/O) cosmetic material but also into an aqueous cosmetic material, such as an oil-in-water (O/W) cosmetic material, skin toner or sun-screening gel, the formulation of which was difficult in the related art. Therefore, it is possible to increase the degree of freedom for the formulation of the cosmetic material.

Furthermore, since the cosmetic material contains the phosphate surfactant, it is possible to make the resin particles into as fine particles as 1 μm or less, and to enhance the ultraviolet-shielding effect.

In addition, according to the cosmetic material of the embodiment, it is possible to obtain a cosmetic material that is excellent in terms of the ultraviolet-shielding function, transparency, a feeling of using the product and safety.

Furthermore, when the cosmetic material of the embodiment is used as a component of a cosmetic product, it is possible to provide a variety of cosmetic products, such as skin care cosmetic products, makeup cosmetic products and body care cosmetic products, which are excellent in terms of the ultraviolet-shielding function, transparency, a feeling of using the product and safety. Particularly, in a case where the cosmetic material is used for whitening by skin care cosmetic products, base-making by makeup cosmetic products and sun-screening by body care cosmetic products, for which an ultraviolet-shielding function is required, it is possible to provide a cosmetic product which is excellent in terms of the ultraviolet-shielding function, transparency, a feeling of using the product and safety.

Meanwhile, the organic ultraviolet absorbent-containing resin particles of the embodiment, a dispersion liquid, the aqueous dispersion element and an oil-based dispersion element, all of which include the resin particles, can also be used for a weather-resistant paint and the like which needs to have the ultraviolet-shielding function.

In addition, in a case where the organic ultraviolet absorbent-containing resin particles, the dispersion liquid, the aqueous dispersion element and the oil-based dispersion element are used in fields other than cosmetic products, there are many cases in which a rough feeling, a feeling of using the product and the like, all of which are emphasized for cosmetic products, do not become serious problems, the dispersant or the resin can be selected more flexibly, and it is possible to increase the degree of freedom for the design and blending of paints and the like.

### EXAMPLES

Hereinafter, the invention will be specifically described using examples and comparative examples, but the invention is not limited by the examples.

A. Production and Evaluation of Resin Monomer-Dissolved Liquids to Moisture Gels

### Example 1

“Production and Evaluation of a Resin Monomer-Dissolved Liquid”

A dibenzoyl methane-based compound (avobenzone, PARSOL (registered trademark) 1789, 6 parts by mass (5.0% by mass)) and a phosphate surfactant (6 parts by mass (5.0% by mass)) were added to methyl methacrylate (108 parts by mass), and fully dissolved, thereby producing a resin monomer-dissolved liquid of Example 1, which contained 5% by mass of the dibenzoyl methane-based compound (avobenzone, PARSOL (registered trademark) 1789).

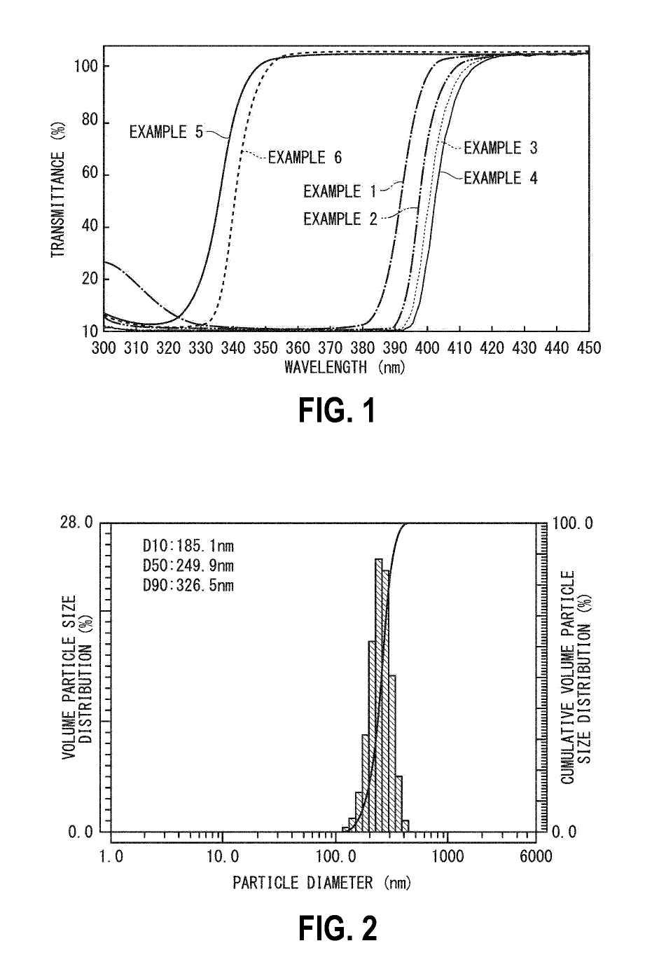

The obtained resin monomer-dissolved liquid was coated on a silica plate in an amount of 2 mg/cm², and the spectral transmittance of the resin monomer-dissolved liquid was measured using an SPF analyzer UV-1000S (manufactured by Labsphere INC. USA). The spectral transmittance is illustrated in FIG. 1.

“Production of an Emulsion”

Pure water (229.5 parts by mass), sodium dodecylbenzosulfonate (0.5 parts by mass), ethylene glycol dimethacrylate (14.0 parts by mass) and a silicone-based defoamer (1.0 part by mass) were mixed with the above resin monomer-dissolved liquid (105 parts by mass) and stirred using a homogenizer, thereby producing an emulsion.

“Production and Evaluation of Resin Particles”

The above emulsion (320.0 parts by mass), pure water (79.856 parts by mass) and potassium persulfate (0.144 parts by mass) were mixed, moved into a reaction apparatus having a stirrer and a thermometer, and subjected to nitrogen substitution for 1 hour.

Next, the nitrogen-substituted reaction solution was heated to 65° C., held at 65° C. for 3 hours, and a polymerization reaction was caused. After that, the solution was cooled using ice to stop the polymerization reaction, the obtained polymer was washed using 2-propanol and pure water, and then dried at 90° C., thereby producing organic ultraviolet absorbent-containing resin particles including polymethyl methacryloate (PMMA) having 5% by mass of avobenzone.

### Example 2

“Production and Evaluation of the Resin Monomer-Dissolved Liquid”

A dibenzoyl methane-based compound (avobenzone, PARSOL (registered trademark) 1789, 12 parts by mass (10.0% by mass)) and a phosphate surfactant (6 parts by mass (5.0% by mass)) were added to methyl methacrylate (102 parts by mass), and fully dissolved, thereby producing a resin monomer-dissolved liquid of Example 2, which contained 10% by mass of the dibenzoyl methane-based compound (avobenzone, PARSOL (registered trademark) 1789).

The spectral transmittance of the obtained resin monomer-dissolved liquid was measured in the same manner as in Example 1. The spectral transmittance is illustrated in FIG. 1.

“Production of an Emulsion”

Pure water (229.5 parts by mass), sodium dodecylbenzosulfonate (0.5 parts by mass), ethylene glycol dimethacrylate (14.0 parts by mass) and a silicone-based defoamer (1.0 part by mass) were mixed with the above resin monomer-dissolved liquid (105 parts by mass) and stirred using a homogenizer, thereby producing an emulsion.

“Production and Evaluation of Resin Particles”

The above emulsion (320.0 parts by mass), pure water (79.856 parts by mass) and potassium persulfate (0.144 parts by mass) were mixed, moved into a reaction apparatus having a stirrer and a thermometer, and subjected to nitrogen substitution for 1 hour.

Next, the nitrogen-substituted reaction solution was heated to 65° C., held at 65° C. for 3 hours, and a polymerization reaction was caused. After that, the solution was cooled using ice to stop the polymerization reaction, the obtained polymer was washed using 2-propanol and pure water, and then dried at 90° C., thereby producing organic ultraviolet absorbent-containing resin particles including polymethyl methacryloate (PMMA) having 10% by mass of the dibenzoyl methane-based compound (avobenzone, PARSOL (registered trademark) 1789).

Next, the dispersed particle diameter of the organic ultraviolet absorbent-containing resin particles was measured using a dynamic light scattering nanoparticle size analyzer LB-550 (manufactured by Horiba, Ltd.). Here, the organic ultraviolet absorbent-containing resin particles (5 parts by mass) was added into a solution, in which polyether-modified silicone (dispersant, 10 parts by mass) and decamethyl cyclopentasiloxane (cyclic silicone, 85 parts by mass) were mixed, and dispersed using a disperser, the dispersed particle diameter of the organic ultraviolet absorbent-containing resin particle dispersion liquid including 5% by mass of the resin particles was measured, and the volume particle size distribution and cumulative volume particle size distribution were computed.

As a result, the particle diameter at 10 volume % (D10) in the cumulative volume particle size distribution was 185.1 nm, the particle diameter at 50 volume % (D50) was 249.9 nm, and the particle diameter at 90 volume % (D90) was 326.5 nm.

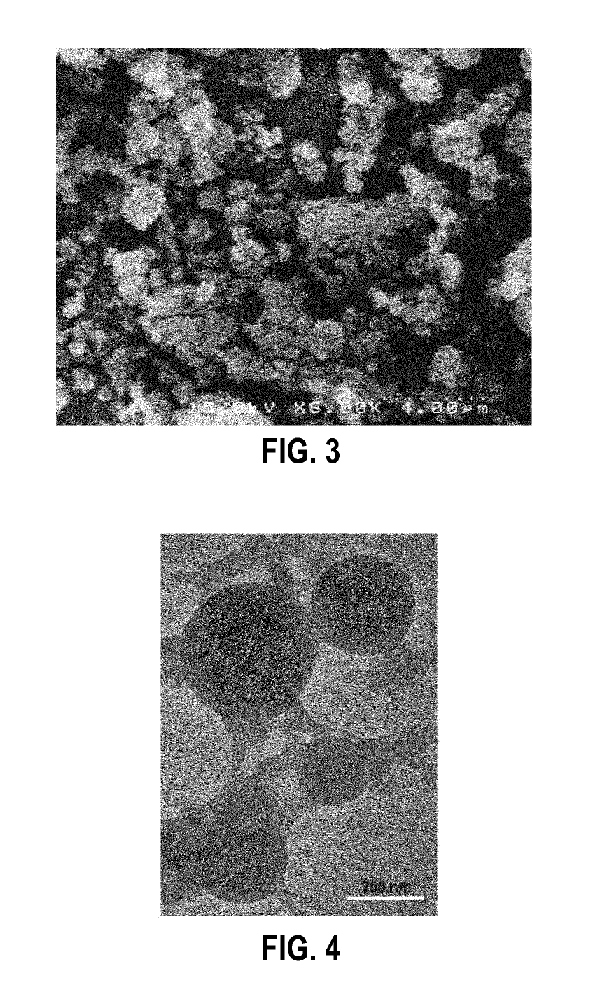

The volume particle size distribution and cumulative volume particle size distribution of the organic ultraviolet absorbent-containing resin particle dispersion liquid are illustrated in FIG. 2. In addition, a scanning electron microscopic (SEM) image of the above organic ultraviolet absorbent-containing resin particles is illustrated in FIG. 3, and a transmission electron microscopic (TEM) image of the organic ultraviolet absorbent-containing resin particles is illustrated in FIG. 4 respectively.

“Production of an Organic Ultraviolet Absorbent-Containing Resin Particle Aqueous Dispersion Element”

The above organic ultraviolet absorbent-containing resin particles (avobenzone 10% by mass+PMMA, 20 parts by mass (40% by mass)), ethanol (7.5 parts by mass (15% by mass)) and pure water (22.5 parts by mass (45% by mass)) were mixed, thereby producing an organic ultraviolet absorbent-containing resin particle aqueous dispersion element including 40% by mass of the resin particles.

“Production of a Moisture Gel using the Organic Ultraviolet Absorbent-Containing Resin Particles”

Sodium carboxy methyl cellulose (1.5 parts by mass (3% by mass)), ethanol (8.13 parts by mass (16.25% by mass)) and glycerin (2.5 parts by mass (5% by mass)) were mixed. Next, the above organic ultraviolet absorbent-containing resin particle aqueous dispersion element (12.5 parts by mass (25.0% by mass)) and pure water (25.38 parts by mass (50.75% by mass)) were mixed, and stirred at 70° C. for 10 minutes under a condition of heating, thereby producing a moisture gel containing 10% by mass of the organic ultraviolet absorbent-containing resin particles (avobenzone 10% by mass+PMMA).

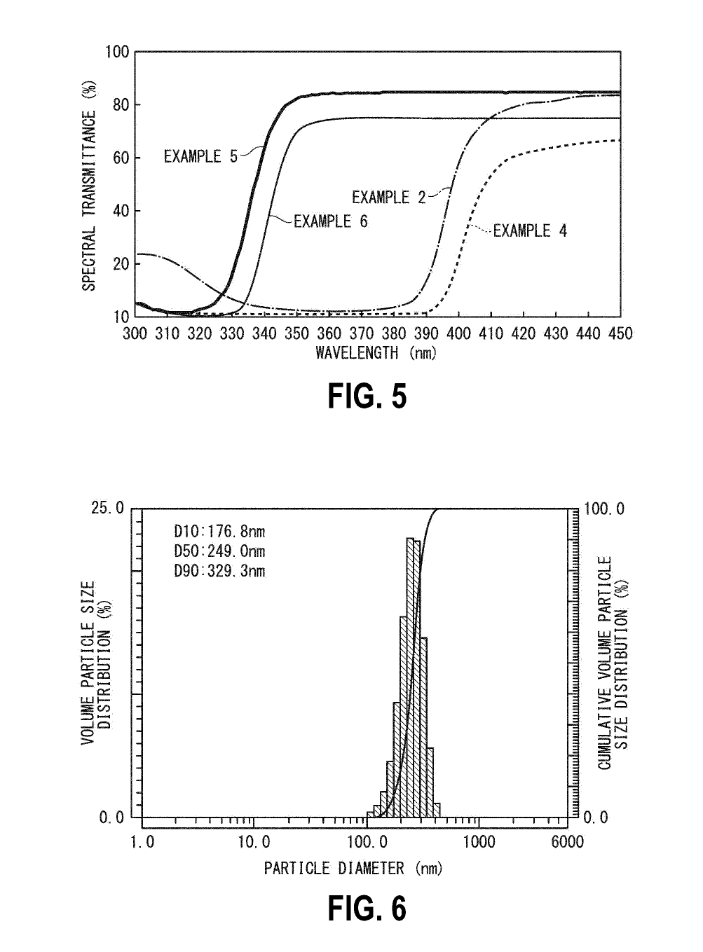

The spectral transmittance and SPF value of the moisture gel were measured in the same manner as in Example 1. The spectral transmittance is illustrated in FIG. 5.

In addition, the viscosity of the moisture gel was measured using a B-type viscometer (manufactured by Toki Sangyo Co., Ltd.), and was 3,500 mPa·s.

### Example 3

“Production and Evaluation of a Resin Monomer-Dissolved Liquid”

A dibenzoyl methane-based compound (avobenzone, PARSOL (registered trademark) 1789, 24 parts by mass (20.0% by mass)) and a phosphate surfactant (6 parts by mass (5.0% by mass)) were added to methyl methacrylate (90 parts by mass), and fully dissolved, thereby producing a resin monomer-dissolved liquid of Example 3, which contained 20% by mass of the dibenzoyl methane-based compound (avobenzone, PARSOL (registered trademark) 1789).

The spectral transmittance of the obtained resin monomer-dissolved liquid was measured in the same manner as in Example 1. The spectral transmittance is illustrated in FIG. 1.

Organic ultraviolet absorbent-containing resin particles including polymethyl methacrylate having 20% by mass of avobenzone were produced in the same manner as in Example 1 except that the above resin monomer-dissolved liquid was used instead of the above resin monomer-dissolved liquid of Example 1.

### Example 4

“Production and Evaluation of a Resin Monomer-Dissolved Liquid”

A dibenzoyl methane-based compound (avobenzone, PARSOL (registered trademark) 1789, 36 parts by mass (30.0% by mass)) and a phosphate surfactant (6 parts by mass (5.0% by mass)) were added to methyl methacrylate (78 parts by mass), and fully dissolved, thereby producing a resin monomer-dissolved liquid of Example 4, which contained 30% by mass of the dibenzoyl methane-based compound (avobenzone, PARSOL (registered trademark) 1789).

The spectral transmittance of the obtained resin monomer-dissolved liquid was measured in the same manner as in Example 1. The spectral transmittance is illustrated in FIG. 1.

“Production of an Emulsion”

An emulsion was produced in the same manner as in Example 2 except that a resin monomer-dissolved liquid containing 30% by mass of the above dibenzoyl methane-based compound (avobenzone, PARSOL (registered trademark) 1789) was used.

“Production and Evaluation of Resin Particles”

The above emulsion (320.0 parts by mass), pure water (79.856 parts by mass) and potassium persulfate (0.144 parts by mass) were mixed, moved into a reaction apparatus having a stirrer and a thermometer, and subjected to nitrogen substitution for 1 hour.

Next, the nitrogen-substituted reaction solution was heated to 65° C., held at 65° C. for 3 hours, and a polymerization reaction was caused. After that, the solution was cooled using ice to stop the polymerization reaction, the obtained polymer was washed using 2-propanol and pure water, and then dried at 90° C., thereby producing organic ultraviolet absorbent-containing resin particles including polymethyl methacryloate (PMMA) having 30% by mass of the dibenzoyl methane-based compound (avobenzone, PARSOL (registered trademark) 1789).

The dispersed particle diameter of the obtained organic ultraviolet absorbent-containing resin particles was measured in the same manner as in Example 2. Here, an organic ultraviolet absorbent-containing resin particle dispersion liquid including 5% by mass of the resin particles was produced using the organic ultraviolet absorbent-containing resin particles, the dispersed particle diameter of the dispersion liquid was measured, and the volume particle size distribution and cumulative volume particle size distribution were computed.

As a result, the particle diameter at 10 volume % (D10) in the cumulative volume particle size distribution was 176.8 nm, the particle diameter at 50 volume % (D50) was 249.0 nm, and the particle diameter at 90 volume % (D90) was 329.3 nm.

The volume particle size distribution and cumulative volume particle size distribution of the organic ultraviolet absorbent-containing resin particle dispersion liquid are illustrated in FIG. 6.

“Production of an Organic Ultraviolet Absorbent-Containing Resin Particle Aqueous Dispersion Element”

An organic ultraviolet absorbent-containing resin particle aqueous dispersion element of Example 4, which included 40% by mass of the resin particles, was produced in the same manner as in Example 2 except that the above organic ultraviolet absorbent-containing resin particles (avobenzone 30% by mass+PMMA) were used instead of the organic ultraviolet absorbent-containing resin particles (avobenzone 10% by mass+PMMA) of Example 2.

“Production of a Moisture Gel using the Organic Ultraviolet Absorbent-Containing Resin Particles”

A moisture gel containing 10% by mass of the organic ultraviolet absorbent-containing resin particles (avobenzone 30% by mass+PMMA) of Example 4 was obtained in the same manner as in Example 2 except that the above organic ultraviolet absorbent-containing resin particles (avobenzone 30% by mass+PMMA) were used instead of the organic ultraviolet absorbent-containing resin particles (avobenzone 10% by mass+PMMA) of Example 2.

The spectral transmittance and SPF value of the moisture gel were measured in the same manner as in Example 1. The spectral transmittance is illustrated in FIG. 5.

In addition, the viscosity of the moisture gel was measured using a B-type viscometer (manufactured by Toki Sangyo Co., Ltd.), and was 3,500 mPa·s.

### Example 5

“Production and Evaluation of a Resin Monomer-Dissolved Liquid”

A resin monomer-dissolved liquid of Example 5 was produced in the same manner as in Example 1 except that ethylhexyl methoxycinnamate (PARSOL (registered trademark) MCX, 6 parts by mass (5.0% by mass)) and the phosphate surfactant (6 parts by mass (5.0% by mass)) were added to methyl methacrylate (108 parts by mass).

The spectral transmittance of the obtained resin monomer-dissolved liquid was measured in the same manner as in Example 1. The spectral transmittance is illustrated in FIG. 1.

“Production of an Emulsion”

An emulsion was produced in the same manner as in Example 2 except that the above resin monomer-dissolved liquid containing 5% by mass of the ethylhexyl methoxycinnamate (PARSOL (registered trademark) MCX) was used.

“Production and Evaluation of Resin Particles”

Organic ultraviolet absorbent-containing resin particles of Example 5, which included polymethyl methacrylate (PMMA) having 5% by mass of the ethylhexyl methoxycinnamate (PARSOL (registered trademark) MCX), were produced in the same manner as in Example 2 except that the above emulsion was used.

“Production of an Organic Ultraviolet Absorbent-Containing Resin Particle Aqueous Dispersion Element”

An organic ultraviolet absorbent-containing resin particles aqueous dispersion element of Example 5, which included 40% by mass of the resin particles, was produced in the same manner as in Example 2 except that the above organic ultraviolet absorbent-containing resin particles (MCX 5% by mass+PMMA) were used instead of the organic ultraviolet absorbent-containing resin particles (avobenzone 10% by mass+PMMA) of Example 2.

A moisture gel of Example 5, which contained 10% by mass of the organic ultraviolet absorbent-containing resin particles (MCX 5% by mass+PMMA) of Example 5, was obtained in the same manner as in Example 2 except that the above organic ultraviolet absorbent-containing resin particles (MCX 5% by mass+PMMA) aqueous dispersion element were used instead of the organic ultraviolet absorbent-containing resin particle (avobenzone 10% by mass+PMMA) aqueous dispersion element of Example 2.

The spectral transmittance and SPF value of the moisture gel were measured in the same manner as in Example 1. The spectral transmittance is illustrated in FIG. 5.

In addition, the viscosity of the moisture gel was measured using a B-type viscometer (manufactured by Toki Sangyo Co., Ltd.), and was 3,600 mPa·s.

### Example 6

“Production and Evaluation of a Resin Monomer-Dissolved Liquid”

A resin monomer-dissolved liquid of Example 6 was produced in the same manner as in Example 1 except that ethylhexyl methoxycinnamate (PARSOL (registered trademark) MCX, 12 parts by mass (10.0% by mass)) and the phosphate surfactant (6 parts by mass (5.0% by mass)) were added to methyl methacrylate (102 parts by mass).

The spectral transmittance of the obtained resin monomer-dissolved liquid was measured in the same manner as in Example 1. The spectral transmittance is illustrated in FIG. 1.

“Production of an Emulsion”

An emulsion was produced in the same manner as in Example 2 except that a resin monomer-dissolved liquid containing 10% by mass of the above ethylhexyl methoxycinnamate (PARSOL (registered trademark) MCX) was used.

“Production and Evaluation of Organic Ultraviolet Absorbent-Containing Resin Particles”

Organic ultraviolet absorbent-containing resin particles of Example 6, which included polymethyl methacrylate (PMMA) having 10% by mass of the ethylhexyl methoxycinnamate (PARSOL (registered trademark) MCX), were produced in the same manner as in Example 2 except that the above emulsion was used.

“Production of an Organic Ultraviolet Absorbent-Containing Resin Particle Aqueous Dispersion Element”

An organic ultraviolet absorbent-containing resin particles aqueous dispersion element of Example 6, which included 40% by mass of the resin particles, was produced in the same manner as in Example 2 except that the above organic ultraviolet absorbent-containing resin particles (MCX 10% by mass+PMMA) were used instead of the organic ultraviolet absorbent-containing resin particles (avobenzone 10% by mass+PMMA) of Example 2.

“Production of a Moisture Gel using the Organic Ultraviolet Absorbent-Containing Resin Particles”

A moisture gel of Example 6, which contained 10% by mass of the organic ultraviolet absorbent-containing resin particles (MCX 10% by mass+PMMA) of Example 6, was obtained in the same manner as in Example 2 except that the above organic ultraviolet absorbent-containing resin particle (MCX 10% by mass+PMMA) aqueous dispersion element was used instead of the organic ultraviolet absorbent-containing resin particle (avobenzone 10% by mass+PMMA) aqueous dispersion element of Example 2.

The spectral transmittance and SPF value of the moisture gel were measured in the same manner as in Example 1. The spectral transmittance is illustrated in FIG. 5.

In addition, the viscosity of the moisture gel was measured using a B-type viscometer (manufactured by Toki Sangyo Co., Ltd.), and was 3,600 mPa·s.

### Comparative Example 1

“Production of a Resin Monomer-Dissolved Liquid”

A dibenzoyl methane-based compound (avobenzone, PARSOL (registered trademark) 1789, 12 parts by mass (10.0% by mass)) was added to methyl methacrylate (108 parts by mass), and fully dissolved, thereby producing a resin monomer-dissolved liquid, which contained 10% by mass of the dibenzoyl methane-based compound (avobenzone, PARSOL (registered trademark) 1789), but did not contain the phosphate surfactant.

“Production of an Emulsion”

An emulsion was produced in the same manner as in Example 2 except that the resin monomer-dissolved liquid, which contained 10% by mass of the dibenzoyl methane-based compound (avobenzone, PARSOL (registered trademark) 1789), but did not contain the phosphate surfactant, was used instead of the resin monomer-dissolved liquid containing 10% by mass of the dibenzoyl methane-based compound (avobenzone, PARSOL (registered trademark) 1789).

“Production and Evaluation of Resin Particles”

The above emulsion (320.0 parts by mass), pure water (79.856 parts by mass) and potassium persulfate (0.144 parts by mass) were mixed, moved into a reaction apparatus having a stirrer and a thermometer, and subjected to a nitrogen substitution for 1 hour.

Next, the nitrogen-substituted reaction solution was heated to 65° C., held at 65° C. for 3 hours, and a polymerization reaction was caused. After that, the solution was cooled using ice to stop the polymerization reaction, the obtained polymer was washed using 2-propanol and pure water, and then, dried at 90° C., thereby producing organic ultraviolet absorbent-containing resin particles (dibenzoyl methane 10% by mass+PMMA) including polymethyl methacrylate (PMMA), which contained 10% by mass of the dibenzoyl methane-based compound (avobenzone, PARSOL (registered trademark) 1789), but did not contain the phosphate surfactant.

The dispersed particle diameter of the obtained organic ultraviolet absorbent-containing resin particles was measured in the same manner as in Example 2.

As a result, the particle diameter at 10 volume % (D10) in the cumulative volume particle size distribution was 561.5 nm, the particle diameter at 50 volume % (D50) was 757.3 nm, and the particle diameter at 90 volume % (D90) was 972.1 nm.

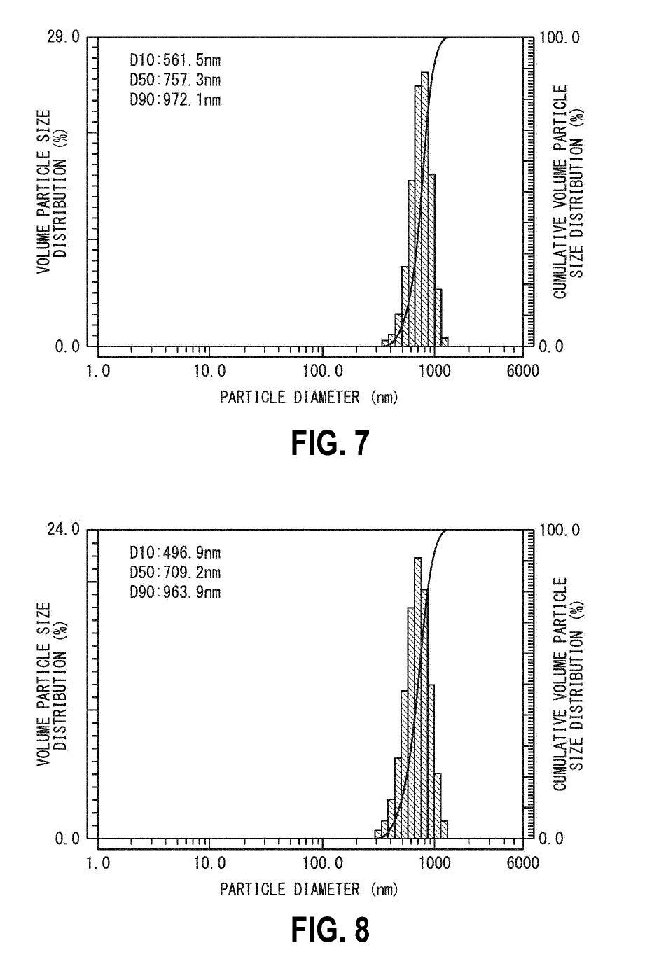

The volume particle size distribution and cumulative volume particle size distribution of the organic ultraviolet absorbent-containing resin particle dispersion liquid are illustrated in FIG. 7.

### Comparative Example 2

“Production of a Resin Monomer-Dissolved Liquid”

A dibenzoyl methane-based compound (avobenzone, PARSOL (registered trademark) 1789, 36 parts by mass (30.0% by mass)) was added to methyl methacrylate (84 parts by mass), and fully dissolved, thereby producing a resin monomer-dissolved liquid, which contained 30% by mass of the dibenzoyl methane-based compound (avobenzone, PARSOL (registered trademark) 1789), but did not contain the phosphate surfactant.

“Production of an Emulsion”

An emulsion was produced in the same manner as in Example 2 except that the resin monomer-dissolved liquid, which contained 30% by mass of the dibenzoyl methane-based compound (avobenzone, PARSOL (registered trademark) 1789), but did not contain the phosphate surfactant, was used instead of the resin monomer-dissolved liquid containing 10% by mass of the dibenzoyl methane-based compound (avobenzone, PARSOL (registered trademark) 1789).

“Production and Evaluation of Resin Particles”

The above emulsion (320.0 parts by mass), pure water (79.856 parts by mass) and potassium persulfate (0.144 parts by mass) were mixed, moved into a reaction apparatus having a stirrer and a thermometer, and subjected to a nitrogen substitution for 1 hour.

Next, the nitrogen-substituted reaction solution was heated to 65° C., held at 65° C. for 3 hours, and a polymerization reaction was caused. After that, the solution was cooled using ice to stop the polymerization reaction, the obtained polymer was washed using 2-propanol and pure water, and then, dried at 90° C., thereby producing organic ultraviolet absorbent-containing resin particles (dibenzoyl methane 30% by mass+PMMA) including polymethyl methacrylate (PMMA), which contained 30% by mass of the dibenzoyl methane-based compound (avobenzone, PARSOL (registered trademark) 1789), but did not contain the phosphate surfactant.

The dispersed particle diameter of the obtained organic ultraviolet absorbent-containing resin particles was measured in the same manner as in Example 2.

As a result, the particle diameter at 10 volume % (D10) in the cumulative volume particle size distribution was 496.9 nm, the particle diameter at 50 volume % (D50) was 709.2 nm, and the particle diameter at 90 volume % (D90) was 963.9 nm.

The volume particle size distribution and cumulative volume particle size distribution of the organic ultraviolet absorbent-containing resin particle dispersion liquid are illustrated in FIG. 8.

B. Production and Evaluation of Simple Sunscreens using the Organic Ultraviolet Absorbent-Containing Resin Particles

### Example 7

“Production of an Oil-Based Dispersion Element of the Organic Ultraviolet Absorbent-Containing Resin Particles”

The organic ultraviolet absorbent-containing resin particles (avobenzone 10% by mass+PMMA, 36 parts by mass) obtained in Example 2, decamethyl cyclopentasiloxane (D5) SH245 (manufactured by Dow Corning Toray Co., Ltd., 75 parts by mass) and polyether-modified silicone SH3775M (manufactured by Dow Corning Toray Co., Ltd., 9 parts by mass) were mixed, and dispersed at 2500 rpm for 3 hours using a sand mill, thereby producing an organic ultraviolet absorbent-containing resin particles (avobenzone 10% by mass+PMMA)-containing oil-based dispersion element of Example 7, which included 30% by mass of the organic ultraviolet absorbent-containing resin particles (avobenzone 10% by mass+PMMA).

“Production and Evaluation of a Simple Sunscreen using the Organic Ultraviolet Absorbent-Containing Resin Particles”

The above organic ultraviolet absorbent-containing resin particle (avobenzone 10% by mass+PMMA)-containing oil-based dispersion element (66.8 parts by mass), linear polyether-modified silicone KF6028 (manufactured by Shin-Etsu Chemical Co., Ltd., 9.6 parts by mass) as a film-forming agent and branched polyether-modified silicone KF6017 (manufactured by Shin-Etsu Chemical Co., Ltd., 10.4 parts by mass) as an emulsifier were well mixed using a mortar with an aqueous solution, in which pure water (8.2 parts by mass) and 1,3-butanediol (5.0 parts by mass) had been mixed in advance, thereby producing a simple sunscreen of Example 7, which contained 20% by mass of the organic ultraviolet absorbent-containing resin particles (avobenzone 10% by mass+PMMA).

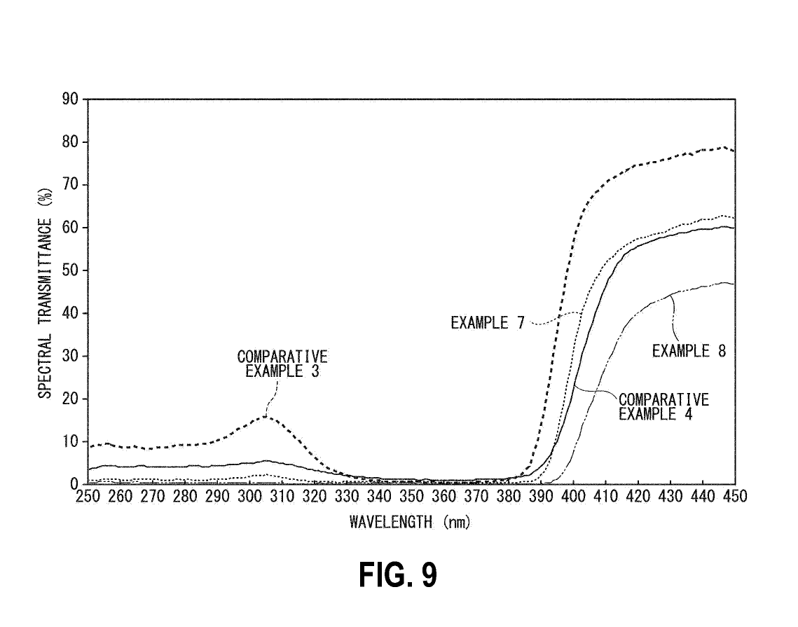

The spectral transmittance of the obtained simple sunscreen was measured in the same manner as in Example 1. The spectral transmittance of the simple sunscreen is illustrated in FIG. 9.

In addition, the SPF value of the simple sunscreen was 70.13.

### Example 8

“Production of an Oil-Based Dispersion Element of the Organic Ultraviolet Absorbent-Containing Resin Particles”

An organic ultraviolet absorbent-containing resin particle (avobenzone 30% by mass+PMMA)-containing oil-based dispersion element of Example 8, which included 30% by mass of the organic ultraviolet absorbent-containing resin particles (avobenzone 30% by mass+PMMA), was produced in the same manner as in Example 7 except that the above organic ultraviolet absorbent-containing resin particles (avobenzone 30% by mass+PMMA) obtained in Example 4 were used instead of the organic ultraviolet absorbent-containing resin particles (avobenzone 10% by mass+PMMA).

“Production and Evaluation of a Simple Sunscreen using the Organic Ultraviolet Absorbent-Containing Resin Particles”

A simple sunscreen of Example 8, which contained 20% by mass of the organic ultraviolet absorbent-containing resin particles (avobenzone 30% by mass+PMMA), was produced in the same manner as in Example 7 except that the above organic ultraviolet absorbent-containing resin particle (avobenzone 30% by mass+PMMA)-containing oil-based dispersion element was used instead of the organic ultraviolet absorbent-containing resin particle (avobenzone 10% by mass+PMMA)-containing oil-based dispersion element.

The spectral transmittance of the obtained simple sunscreen was measured in the same manner as in Example 1. The spectral transmittance of the simple sunscreen is illustrated in FIG. 9.

In addition, the SPF value of the simple sunscreen was 288.57.

### Comparative Example 3

“Production of an Oil-Based Dispersion Element of the Organic Ultraviolet Absorbent-Containing Resin Particles”

An organic ultraviolet absorbent-containing resin particle (avobenzone 10% by mass+PMMA)-containing oil-based dispersion element of Comparative Example 3 not containing the phosphate surfactant, which included 30% by mass of the organic ultraviolet absorbent-containing resin particles (avobenzone 10% by mass+PMMA) and does not contain the phosphate surfactant, was produced in the same manner as in Example 7 except that the above organic ultraviolet absorbent-containing resin particles (avobenzone 10% by mass+PMMA) of Comparative Example 1 not containing the phosphate surfactant were used instead of the organic ultraviolet absorbent-containing resin particles (avobenzone 10% by mass+PMMA).

“Production and Evaluation of a Simple Sunscreen using the Organic Ultraviolet Absorbent-Containing Resin Particles”

A simple sunscreen of Comparative Example 3, which contained 20% by mass of the organic ultraviolet absorbent-containing resin particles (avobenzone 10% by mass+PMMA) including polymethyl methacrylate (PMMA) and does not contain the phosphate surfactant, was produced in the same manner as in Example 7 except that the above organic ultraviolet absorbent-containing resin particle (avobenzone 10% by mass+PMMA)-containing oil-based dispersion element, which included polymethyl methacrylate (PMMA) and does not contain the phosphate surfactant, was used instead of the organic ultraviolet absorbent-containing resin particle (avobenzone 10% by mass+PMMA)-containing oil-based dispersion element.

The spectral transmittance of the obtained simple sunscreen was measured in the same manner as in Example 1. The spectral transmittance of the simple sunscreen is illustrated in FIG. 9.

In addition, the SPF value of the simple sunscreen was 8.37.

### Comparative Example 4

“Production of an Oil-Based Dispersion Element of the Organic Ultraviolet Absorbent-Containing Resin Particles”

An organic ultraviolet absorbent-containing resin particle (avobenzone 30% by mass+PMMA)-containing oil-based dispersion element of Comparative Example 4 not containing the phosphate surfactant, which included 30% by mass of the organic ultraviolet absorbent-containing resin particles (avobenzone 30% by mass+PMMA) not containing the phosphate surfactant, was produced in the same manner as in Example 7 except that the organic ultraviolet absorbent-containing resin particles (avobenzone 30% by mass+PMMA) of Comparative Example 2 not containing the phosphate surfactant were used instead of the organic ultraviolet absorbent-containing resin particles (avobenzone 10% by mass+PMMA).

“Production and Evaluation of a Simple Sunscreen using the Organic Ultraviolet Absorbent-Containing Resin Particles”

A simple sunscreen of Comparative Example 4, which contained 20% by mass of the organic ultraviolet absorbent-containing resin particles (avobenzone 30% by mass+PMMA) including polymethyl methacrylate (PMMA) and does not containing the phosphate surfactant, was produced in the same manner as in Example 7 except that the above organic ultraviolet absorbent-containing resin particle (avobenzone 30% by mass+PMMA)-containing oil-based dispersion element, which includes polymethyl methacrylate (PMMA) and does not contain the phosphate surfactant, was used instead of the organic ultraviolet absorbent-containing resin particle (avobenzone 10% by mass+PMMA)-containing oil-based dispersion element.

The spectral transmittance of the obtained simple sunscreen was measured in the same manner as in Example 1. The spectral transmittance of the simple sunscreen is illustrated in FIG. 9.

In addition, the SPF value of the simple sunscreen was 22.33.

The respective SPF values and critical wavelengths of Examples 7 and 8, and Comparative Examples 3 and 4 will be described in Table 1.

TABLE 1

| | SPF value | Critical wavelength (nm) |
|---|---|---|
| Example 7 | 70.13 | 386 |
| Example 8 | 288.57 | 393 |
| Comparative Example 3 | 8.37 | 383 |
| Comparative Example 4 | 22.33 | 392 |

When the volume particle size distributions and cumulative volume particle size distributions of the resin particle dispersion liquids of Examples 2 and 4 and the volume particle size distributions and cumulative volume particle size distributions of the resin particle dispersion liquids of Comparative Examples 1 and 2 are compared, it was confirmed that the particle diameter could be decreased by adding the phosphate surfactant.

Although the same amount of the resin particles were blended, ultraviolet rays could be shielded from the vicinity of 390 nm in Examples 7 and 8, and ultraviolet rays could not be shielded from the vicinity of 380 nm in Comparative Examples. Since the phosphate surfactant was mixed so that the resin particle diameter was decreased in Examples 7 and 8, the number of voids among the particles is small when the particles are made into a coated film. Therefore, the transmittance is suppressed to be almost 1% or less from 290 nm to the vicinity of the critical wavelength, and the ultraviolet-shielding effect is high. In contrast, in Comparative Examples 3 and 4, in which the phosphate surfactant was not mixed, since the particle diameter is relatively large, the number of voids among the particles is large in a case where the particles are made into a coated film, and the ultraviolet-shielding effect is low.

It is considered that the high SPF values in Examples 7 and 8 and the low SPF values in Comparative Examples 3 and 4 result from the above reason. Base on the above fact, it was found that the ultraviolet-shielding effect can be enhanced by adding the phosphate surfactant.

### INDUSTRIAL APPLICABILITY

The organic ultraviolet absorbent-containing resin particles of the invention are organic ultraviolet absorbent-containing resin particles including a resin containing an organic ultraviolet absorbent and a phosphate surfactant, and, when the average particle diameter of the organic ultraviolet absorbent-containing resin particles is set to 0.05 μm or more and 1 μm or less, and the content of the organic ultraviolet absorbent is set to 0.1% by mass or more and 80% by mass or less, the ultraviolet-shielding effect is high, the burden for contacted skin can be reduced, and the particles can be safely used. Therefore, the particles can be applied not only to a water-in-oil (W/O) cosmetic product but also to an oil-in-water (O/W) cosmetic product, the degree of freedom for the formulation as a cosmetic material can be improved, and the industrial value is large.

1. Organic ultraviolet absorbent-containing resin particles, comprising: a resin including an organic ultraviolet absorbent and a phosphate surfactant,
wherein an average particle diameter of the organic ultraviolet absorbent-containing resin particles is 0.05 μm or more and 1 μm or less, and a content of the organic ultraviolet absorbent is 0.1% by mass or more and 80% by mass or less.

2. Organic ultraviolet absorbent-containing resin particles, comprising: a resin including an organic ultraviolet absorbent and a phosphate surfactant,
wherein an average particle diameter of the organic ultraviolet absorbent-containing resin particles is 0.05 μm or more and 0.5 μm or less, and a content of the organic ultraviolet absorbent is 0.1% by mass or more and 80% by mass or less.

3. The organic ultraviolet absorbent-containing resin particles according to claim 1,
wherein the organic ultraviolet absorbent is one or two or more selected from the group consisting of dibenzoyl methane-based compounds, benzophenone derivatives, para-aminobenzoic acid derivatives, methoxycinnamic acid derivatives and salicylic acid derivatives.

4. A method for manufacturing organic ultraviolet absorbent-containing resin particles, comprising:
dissolving an organic ultraviolet absorbent and a phosphate surfactant are dissolved in resin monomers so that a content of the organic ultraviolet absorbent becomes 0.1% by mass or more and 80% by mass or less and a content of the phosphate surfactant becomes 1% by mass or more and 50% by mass or less to produce a resin monomer-dissolved liquid;
suspending or emulsifying the resin monomer-dissolved liquid in pure water including a suspension protectant, a silicone-based defoamer and a crosslinking agent to produce a suspended liquid or an emulsified liquid; and
adding a polymerization initiator to the suspended liquid or the emulsified liquid to perform suspension polymerization or emulsion polymerization to generate organic ultraviolet absorbent-containing resin particles.

5. An organic ultraviolet absorbent-containing resin particle dispersion liquid, wherein the organic ultraviolet absorbent-containing resin particles of claim 1 are dispersed in a dispersion medium and a content of the organic ultraviolet absorbent-containing resin particles is 1% by mass or more and 80% by mass or less.

6. An organic ultraviolet absorbent-containing resin particle aqueous dispersion element, wherein the organic ultraviolet absorbent-containing resin particles of claim 1 are dispersed in a dispersion medium including alcohols and a content of the organic ultraviolet absorbent-containing resin particles is 1% by mass or more and 80% by mass or less, and a content of the alcohols is 5% by mass or more and 20% by mass or less.

7. An organic ultraviolet absorbent-containing resin particle oil-based dispersion element, wherein the organic ultraviolet absorbent-containing resin particles of claim 1 are dispersed in an oily component including a surfactant and a content of the organic ultraviolet absorbent-containing resin particles is 1% by mass or more and 80% by mass or less, a content of the oily component is 10% by mass or more and 90% by mass or less, and a content of the surfactant is 1% by mass or more and 40% by mass or less.

8. A cosmetic material, comprising:
1% by mass or more and 60% by mass or less of the organic ultraviolet absorbent-containing resin particles of claim 1.

9. The organic ultraviolet absorbent-containing resin particles according to claim 2,
wherein the organic ultraviolet absorbent is one or two or more selected from the group consisting of dibenzoyl methane-based compounds, benzophenone derivatives, para-aminobenzoic acid derivatives, methoxycinnamic acid derivatives and salicylic acid derivatives.

10. An organic ultraviolet absorbent-containing resin particle dispersion liquid, wherein the organic ultraviolet absorbent-containing resin particles of claim 2 are dispersed in a dispersion medium and a content of the organic ultraviolet absorbent-containing resin particles is 1% by mass or more and 80% by mass or less.

11. An organic ultraviolet absorbent-containing resin particle aqueous dispersion element, wherein the organic ultraviolet absorbent-containing resin particles of claim 2 are dispersed in a dispersion medium including alcohols and a content of the organic ultraviolet absorbent-containing resin particles is 1% by mass or more and 80% by mass or less, and a content of the alcohols is 5% by mass or more and 20% by mass or less.

12. An organic ultraviolet absorbent-containing resin particle oil-based dispersion element, wherein the organic ultraviolet absorbent-containing resin particles of claim 2 are dispersed in an oily component including a surfactant and a content of the organic ultraviolet absorbent-containing resin particles is 1% by mass or more and 80% by mass or less and a content of the oily component is 10% by mass or more and 90% by mass or less, and a content of the surfactant is 1% by mass or more and 40% by mass or less.

13. A cosmetic material, comprising:
1% by mass or more and 60% by mass or less of the organic ultraviolet absorbent-containing resin particles of claim 2.

14. A cosmetic material, comprising:
1% by mass or more and 60% by mass or less of the organic ultraviolet absorbent-containing resin particle dispersion liquid of claim 5 in terms of the organic ultraviolet absorbent-containing resin particles.

15. A cosmetic material, comprising:
1% by mass or more and 60% by mass or less of the organic ultraviolet absorbent-containing resin particle aqueous dispersion element of claim 6 in terms of the organic ultraviolet absorbent-containing resin particles.

16. A cosmetic material, comprising:
1% by mass or more and 60% by mass or less of the organic ultraviolet absorbent-containing resin particle oil-based dispersion element of claim 7 in terms of the organic ultraviolet absorbent-containing resin particles.

17. A cosmetic material, comprising:
1% by mass or more and 60% by mass or less of the organic ultraviolet absorbent-containing resin particle dispersion liquid of claim 10 in terms of the organic ultraviolet absorbent-containing resin particles.

18. A cosmetic material, comprising:
1% by mass or more and 60% by mass or less of the organic ultraviolet absorbent-containing resin particle aqueous dispersion element of claim 11 in terms of the organic ultraviolet absorbent-containing resin particles.

19. A cosmetic material, comprising:
1% by mass or more and 60% by mass or less of the organic ultraviolet absorbent-containing resin particle oil-based dispersion element of claim 12 in terms of the organic ultraviolet absorbent-containing resin particles.
